(19) United States
(12) Patent Application Publication
(10) Pub. No.: US 2015/0305823 A1
(43) Pub. Date: Oct. 29, 2015

(54) SYSTEM AND METHOD FOR PROCESSING NAVIGATIONAL SENSOR DATA

(71) Applicant: General Electric Company, Schenectady, NY (US)

(72) Inventor: Bernhard Erich Hermann Claus, Niskayuna, NY (US)

(73) Assignee: General Electric Company, Schenectady, NY (US)

(21) Appl. No.: 14/262,415

(22) Filed: Apr. 25, 2014

(51) Int. Cl. A61B 19/00; A61B 5/06; G01B 7/00

(52) U.S. Cl. CPC A61B 19/5244; G01B 7/003; A61B 5/061; A61B 2019/5251

(57) ABSTRACT

Aspects of the present disclosure relate to approaches for determining position and orientation of a tracked tool in a medical navigational context. In one embodiment, the position of a surgical or interventional tool may be determined using the orientation or field direction data such that the determination is independent of field strength or magnitude. Feedback may be provided to a user based on these determinations. In certain embodiments, the navigational system may be auto-calibrated using position information determined independent of field strength or magnitude.

### BACKGROUND

The subject matter disclosed herein relates generally to an interventional or surgical navigation system that may be used to provide position and orientation information for an instrument, implant or device used in a medical context, such as in a surgical or interventional context.

In various medical contexts it may be desirable to acquire position and/or orientation information for a medical instrument, implant or device that is navigated or positioned (externally or internally) relative to a patient. For example, in surgical and/or interventional contexts, it may be useful to acquire position and/or orientation information for a medical device when the device, or a relevant portion of the device, is out of view, such as within a patient's body. Likewise, in certain procedures where an imaging technique is used to observe all or part of an interventional or surgical procedure, it may be useful to have position and orientation information derived from the tracked device itself that can be related to the image data also being acquired.

With this in mind, certain navigation systems employ a sensing mechanism (i.e., a sensor) within the navigated instrument. The sensor, when exposed to externally generated electromagnetic fields, generates measurements in response to the local field strengths and orientations. These measurements can then be used in determining the position and orientation of the sensor. However, such systems may be sensitive to calibration errors as well as to calibration drift over time. Further the measurements generated by such sensors may be increasingly noisy as the distance between the sensor and the source of the electromagnetic fields (typically one or more electromagnetic transmission coils) increases.

### BRIEF DESCRIPTION

In one embodiment, a surgical or interventional navigation system is provided. The surgical or interventional navigation system includes a transmitter assembly having at least a first transmitter coil and a second transmitter coil and a sensor assembly having one or more sensor components defining a plane. The surgical or interventional navigation system also includes an electromagnetic tracking system in communication with both the transmitter assembly and the sensor assembly. The electromagnetic tracking system is configured to: acquire a plurality of measurements using the sensor assembly, wherein each measurement corresponds to a projection of a three-dimensional vector onto the plane; and to determine one or both of an orientation and a position of the sensor assembly based on the polar coordinates of the measurements.

In a further embodiment, a surgical or interventional navigation system is provided. The surgical or interventional navigation system includes a transmitter assembly having at least a first transmitter coil and a second transmitter coil and a sensor assembly having one or more sensor components defining a plane. The sensor assembly is configured to generate measurements corresponding to position and orientation of the sensor assembly within electromagnetic fields generated by the transmitter assembly. The surgical or interventional navigation system also includes an electromagnetic tracking system in communication with both the transmitter assembly and the sensor assembly. The electromagnetic tracking system is configured to: drive the first transmitter coil at a first frequency and the second transmitter coil at a second frequency when the sensor assembly is within a one or more of a threshold distance, field strength, or field orientation relative to both the first transmitter coil and the second transmitter coil; and to drive the first transmitter coil in a multiplexed manner at the first frequency and at the second frequency and not drive the second transmitter coil when the sensor assembly is within the threshold distance, field strength, or field orientation relative to the first transmitter coil and outside the threshold distance, field strength, or field orientation relative to the second transmitter coil.

In an additional embodiment, a surgical or interventional navigation system is provided. The surgical or interventional navigation system includes a display, a transmitter assembly having at least a first transmitter coil and a second transmitter coil; and a sensor assembly having one or more sensor components defining a plane. The sensor assembly is configured to generate measurements corresponding to position and orientation of the sensor assembly within electromagnetic fields generated by the transmitter assembly. The surgical or interventional navigation system also includes an electromagnetic tracking system in communication with the transmitter assembly, the sensor assembly, and the display. The electromagnetic tracking system is configured to provide feedback comprising user instructions via the display. The feedback is based on the position and orientation of the sensor assembly within a navigable volume defined by the transmitter assembly and on the expected noise characteristics at the position and orientation.

In another embodiment, a method for operating a medical navigation system is provided. In accordance with this embodiment, a measurement is acquired using a sensor assembly. Sensing elements of the sensor assembly define a planar measurement space and the measurement corresponds to a projection of a three-dimensional vector onto the planar measurement space. The measurement is expressed in polar coordinates. One or both of an orientation and a position of the sensor assembly are determined based on the polar coordinates of the measurement. Operation of the medical navigation system or a procedure being implemented using the medical navigation system is adapted based on the position and orientation of the sensor assembly.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 is a schematic representation of a surgical navigation system in accordance with aspects of the present disclosure;

FIG. 2 depicts an example of an interventional device suitable for use with a position and orientation sensor assembly of FIG. 1, in accordance with aspects of the present disclosure;

FIG. 3 depicts a measurement plane associated with a two-dimensional sensor assembly, in accordance with aspects of the present disclosure;

FIG. 4 depicts decomposition of an error vector, in accordance with aspects of the present disclosure;

FIG. 5 depicts a process flow diagram depicting steps of a calibration approach in accordance with aspects of the present disclosure;

FIG. 6 depicts a process flow diagram depicting steps of a mis-calibration detection approach in accordance with aspects of the present disclosure; and

FIG. 7 depicts a process flow diagram depicting a temporal smoothing approach in accordance with aspects of the present disclosure.

### DETAILED DESCRIPTION

Various approaches are discussed herein for improving the processing algorithms, and systems implementing such algorithms, used in determining position and orientation information for a medical navigation system. By way of example, in one implementation the criterion that is minimized as part of the position and orientation determination is essentially independent of local electromagnetic field strength, thereby making the minimization operation independent of field strength calibration. Alternatively, such an approach may also allow for field strength calibration, including auto-calibration of the navigation sensor. In addition, noise performance of the navigation system may be improved by the approaches discussed herein.

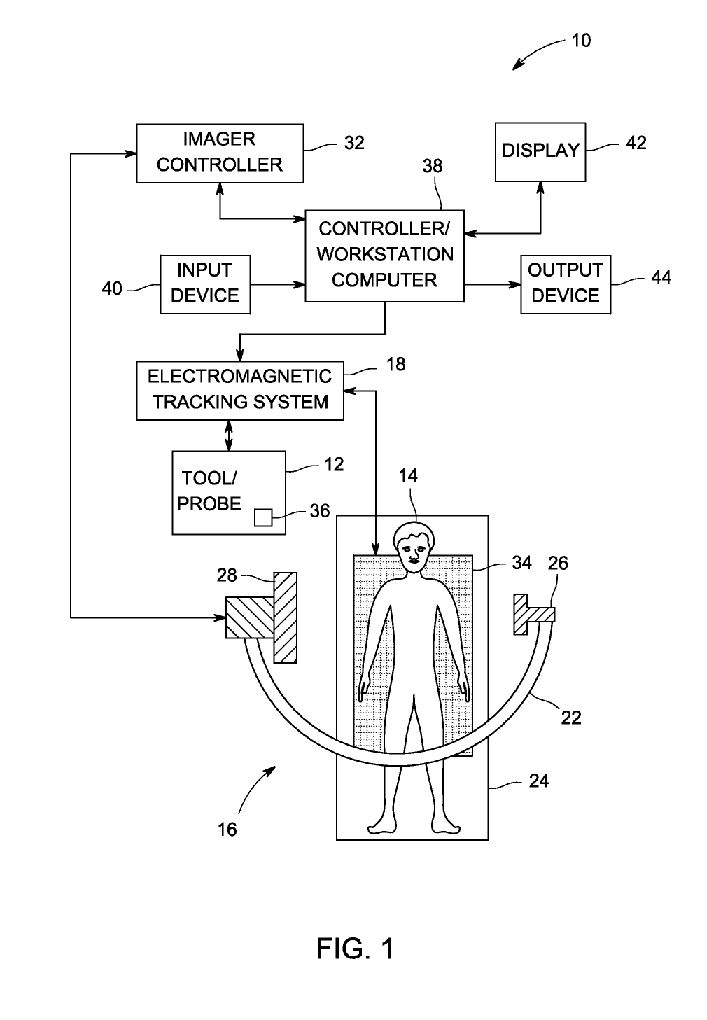

With the preceding in mind, FIG. 1 illustrates an embodiment of a navigation system 10 suitable for tracking movement of a surgical or interventional tool or object 12 (e.g., a catheter, a laparoscope, and so forth) with respect to a patient 14. In the depicted example, the system 10 includes an electromagnetic (EM) tracking system 18 operable to track movement of the tool or object 12 through the volume containing the subject 14.

In one embodiment, the EM tracking system 18 includes a transmitter assembly 34 capable of generating one or more electromagnetic fields in the area in which the patient 14 is positioned. By way of example, the transmitter assembly 34 may include one or more electromagnetic coils positioned near, (e.g., beneath) the imaged subject 14. In one embodiment, the transmitter assembly 34 includes multiple, discrete and separately operable transmitter coils (such as between 2-20 coils (e.g., 10 or 12 coils)), which may each be driven at a different frequency so as to be discernible from one another. In such an embodiment, the spatial arrangement of the coils with respect to one another is typically known and/or fixed. In certain implementations, the transmitter assembly 34 may be a wireless or wired device.

The EM tracking system 18 may also include at least one position and orientation sensor assembly 36 (e.g., a receiver assembly) for generating position information with respect to the electromagnetic fields generated by the transmitter assembly 34. For example, the position and orientation sensor assembly 36 may include one or more EM coils or magnetoresistance (MR) sensors that generate signals indicative of the position and orientation of the respective sensor assembly 36 relative to the electromagnetic fields generated by the transmitter assembly 34. In one implementation, the sensor assembly 36 may generate signals relating to the strength and direction of an electric field based on the position and orientation of the sensor assembly 36 within the electromagnetic field. As with the transmitter assembly 34, the position and orientation sensor assembly 36 may also be a wireless or wired device. In embodiments employing a wireless transmitter assembly 34 or wireless position and orientation sensor assembly 36, separate power units may be provided, such as batteries or photocells, for example.

In one implementation, the position and orientation sensor assembly 36 is located within a tool or probe 12 navigated through the patient 14. Alternatively, in some implementations the position and orientation sensor assembly 36 may be rigidly attached to an internal organ or to the external body of the imaged subject 14 in a conventional manner to provide position and orientation information for the organ or patient 14. In yet another embodiment, a sensor assembly 36 may be attached to a component of the imaging device or system (e.g., a gantry supporting an X-ray source and/or detector) such as to ascertain the position of the imaging device relative to the transmitter assembly and/or the tool or probe (and vice versa). In general, the tool or probe 12 may include a surgical or interventional tool or device to be tracked when navigated through and/or around the subject 14.

In one embodiment, the EM tracking system 18 includes electronics coupled to and communicating with both the transmitter assembly 34 and the position and orientation sensor assembly 36 to determine or calculate the position and orientation of the sensor assembly 36 with respect to the transmitter assembly 34. For example, the EM tracking system 18 may include drive circuitry configured to provide a drive current to each coil of the transmitter assembly 34. In such an embodiment, a drive current may be supplied by the drive circuitry to energize a coil or coils of the transmitter assembly 34, and thereby generate an electromagnetic field that is detected by the position and orientation sensor assembly 36, as discussed herein.

The drive current may be a periodic waveform with a given frequency (e.g., a sinusoidal or other periodic signal). As noted above, different coils may be operated at different frequencies so as to be distinguishable based on their respective frequencies. That is, the drive current supplied to the transmitter coils will generate an electromagnetic field at the same frequency as the drive current. As discussed herein, the respective electromagnetic fields are detectable using the receiver assembly 36 to derive position and orientation information for the tool 12. With this in mind, the EM tracking system 18 may include receiver data acquisition circuitry for receiving signals from the position and orientation sensor assembly 36 and for translating or processing such signals (as discussed herein) to obtain the position and orientation information associated with the tool 12.

In one embodiment, coils of the transmitter assembly 34 may be characterized as single dipole coils that emit magnetic fields when a current is passed through the coils. As noted above, multiple electromagnetic field generating coils may be used in coordination to generate multiple magnetic fields. The position and orientation sensor assembly 36 may employ electromagnetic coils, magnetoresistance sensors, or other suitable components to detect the magnetic fields emitted by the transmitter assembly 34. When a current is applied to the coils of the transmitter assembly 34, one or more magnetic fields are created that encompass at least a portion of the patient undergoing a procedure as well as the vicinity in which the position and orientation sensor assembly 36 will be used. Each field, then, induces a response (such as a responsive current) in the position and orientation sensor assembly 36, which may be measured, sensed, or otherwise detected to generate an output signal for analysis.

The navigation system 10 may further include a controller or workstation computer 38 coupled to and receiving data from the tracking system 18. In embodiments where imaging is employed, as discussed below, the controller 38 may be configured to receive or calculate the position and orientation of the tool 12 relative to acquired imaging data from the imaging system 16. The overall navigation system 10, in such an implementation, is thereby operable to determine the location and orientation of the position and orientation sensor assembly 36 or attached tool 12 relative to the transmitter assembly field, and to correlate this location and orientation to one or more pre-acquired or real-time images acquired by the imaging system 16.

In such an imaging context, the patient 14 may be imaged in conjunction with the surgical or interventional procedure, such as continuously, discontinuously (i.e., as needed), or periodically. In such embodiments, the system 10 includes or communicates with an imaging system 16. In other embodiments, the imaging system 16 may be absent or may be operated separately from the navigation system 10. When present, the imaging system 16 is generally operable to generate a two-dimensional, three-dimensional, or four-dimensional image data corresponding to an area of interest of the imaged subject 14, typically the portion of the patient 14 undergoing the surgical or interventional procedure. Examples of suitable imaging systems 16 include, but are not limited to, computed tomography (CT), C-arm radiography (e.g., angiography), magnetic resonance imaging (MRI), positron emission tomography (PET), computerized tomosynthesis , ultrasound (US), fluoroscopy, and so forth. The imaging system 16 can be operable to generate static images prior to a medical procedure or real-time (or near real-time) images acquired while a procedure (e.g., angioplastic procedures, laparoscopic procedures, endoscopic procedures, etc.) is performed. Thus, the type of images can be diagnostic or interventional. As discussed above, in order to establish the position/orientation of the imaging system relative to the tracked tool, it may also be combined with an additional sensor assembly 36.

In the depicted example, the illustrated imaging system 16 includes a conventional C-arm 22 positioned to direct radiation toward an imaged subject 14 positioned on a surgical table 24. The imaging system further includes a radiation source 26 and a detector 28. A navigation calibration target may be present in some embodiments, such as attached to the detector 28. If present, the calibration target 30 may communicate with the EM tracking system 18 via a cable or wirelessly.

When present, the imaging system 16 may be controlled by, or communicate with, an imager controller 32. Such a controller 32 may be configured to control operation and/or motion of an X-ray source 26 and detector 28, or other imaging subsystems which may vary depending on the imaging modality. For example depending on the imaging protocol, the radiation source 26 and image detector 28 of the imaging system 16 may be selectively moved to and operated at various positions so as to acquire image data (e.g., two-dimensional, three-dimensional) at different views of one or more regions of interest of the medical imaged subject 14, or four-dimensional data (three-dimensional data over a desired time period).

The controller or workstation computer 38 may, in certain embodiments, communicate with and/or control the imager controller 32 as well as the EM tracking system 18 so as to enable each to be in synchronization with one another and facilitate combination of both the acquired image data and navigational data (e.g., position and orientation data for the tool 12). In one embodiment, the controller 38 includes one or more processors as well memory circuitry. The processor can be arranged independent of or integrated with the memory. Although the processor and memory are described as being in the controller 38, it should be understood that the processor or memory, or portions thereof, can be located at the imager controller 32, the EM tracking system 18, or other portions of the system 10 suitable for housing such electronic components. The processor is generally operable to execute program instructions stored within the memory such as algorithms which, when executed, calculate the position (and orientation) of the position and orientation sensor assembly 36 relative to the transmitter assembly 34, or vice versa. The processor can also be capable of receiving input or information or communicating output data. Examples of the processor include a digital signal processor, a central processing unit, or the like.

An embodiment of the memory may include one or more computer-readable media operable to store a plurality of computer-readable program instructions for execution by the processor. The memory can also be operable to store data generated or received by the controller 38. By way of example, such media may include RAM, ROM, PROM, EPROM, EEPROM, flash, CD-ROM, DVD, or other known computer-readable media or combinations thereof which can be used to carry or store desired program code in the form of instructions or data structures and which can be accessed by a general purpose or special purpose computer or other machine with a processor.

Though the controller 38, imager controller 32, and EM tracking system are described separately herein to facilitate explanation of their respective operations and functions, in practice these systems may be provide as a single integrated system with common processing and memory components. Conversely, in other embodiments one or more of these systems may be provided as a separate, stand-alone type component such that the EM tracking system 18 may be combined with different imaging modalities.

In the depicted example, the controller 38 includes, or is in communication with, an input device 40, a display 42, and an output device 44. The input device 40 can be generally operable to receive and communicate information or data from a user to the controller 38. The input device 40 can include a mouse device, pointer, keyboard, touch screen, microphone, or other like device or combinations thereof capable of receiving a user directive. The display 42 is generally operable to illustrate output data for viewing by the user. For example, the display 42 may be used to show static or real-time image data generated using the imaging system 16 with tracking data generated by the EM tracking system 18. The display 42 is capable of illustrating two-dimensional, three dimensional, and/or four-dimensional image data, or a combination thereof, through shading, coloring, and/or the like. Position information may be overlayed on displayed image data. In one embodiment, image data is acquired periodically, and position and orientation information is overlayed in real-time on the most recent available image data, as the catheter/tool is positioned. Additional information about the current state of the EM tracking system may be displayed as well, e.g., position and orientation information, current estimated accuracy, output of failure modes, output for interactive modes as discussed in more detail herein below, etc. Examples of the display 42 include, but are not limited to, a cathode ray monitor, a liquid crystal display (LCD) monitor, a touchscreen monitor, or a plasma monitor. The output device 44 can be generally operable to illustrate or audibilize output data for viewing or for listening, respectively, by the user. The output device 44 can include additional display or screen devices, a visual alarm, an audible alarm, and so forth.

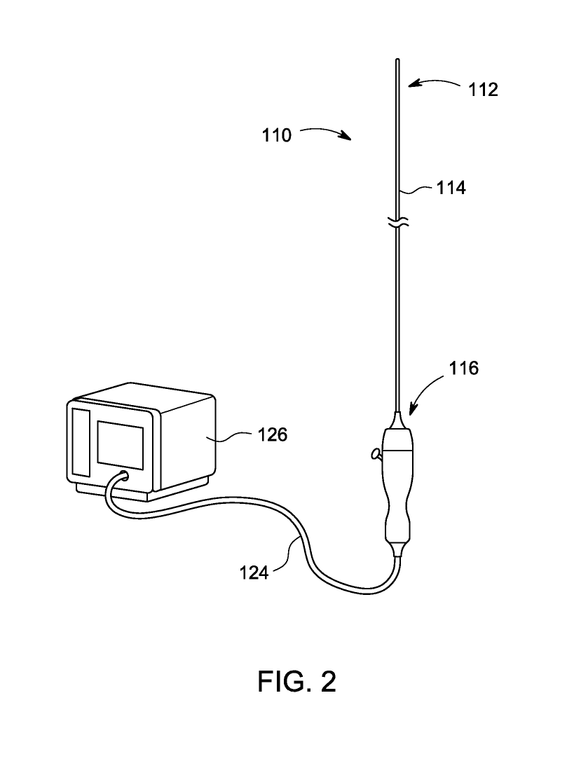

Turning to FIG. 2, an example of a medical device is depicted that is suitable for use with a position and orientation sensor assembly 36 (e.g., receiver assembly) as discussed herein. In this example, the medical device is a catheter 50 suitable for insertion into and navigation through the vasculature of a patient. As will be appreciated, though a catheter is provided as an example, the position and orientation sensor assembly 36 discussed herein may be provided on or in various other types of surgical or interventional instruments, implants or devices. Examples of such instruments, implants or devices include, but are not limited to: a surgical implant, probe, awl, drill, aspirator, forceps, blade, screw, nail, pin, k-wire, needle, cannula, introducer, catheter, guidewire, stent, heart valve, filter, laparoscope, electrode, endoscopes or other intrabody camera devices, or any other suitable device for which position and orientation information may be desired during surgical or interventional use.

Turning back to FIG. 2, the depicted catheter includes a distal end or tip 52 in which the position and orientation sensor assembly 36 may be positioned as well as a shaft 54 in communication with the tip 52 and which connects the tip 52 with a handle assembly 56 that may be used to manipulate and operate the catheter 50. In certain instances, the handle may communicate, such as via cable 58, with an operator console 60 that allows a user to control certain aspects of the catheter function and operation and/or which may provide some or all of the functionality of the EM tracking system 18.

With the preceding discussion in mind, present approaches are described that provide improvement and optimization of position and orientation measurement accuracy in the context of surgical and interventional instrument tracking. Such approaches may be employed to allow self-calibrating navigational systems or processes, temporal integration or smoothing of the measurement data, seed point selection for tracking algorithms, adaptive navigational systems or processes, as well as system feedback that may be useful to a person conducting a navigational procedure. In certain embodiments, a model may be established that models deviations in the acquired signals and the associated position/orientation (e.g., error and/or noise components in the signal or processed data) where portions of the model capture locally linear responses.

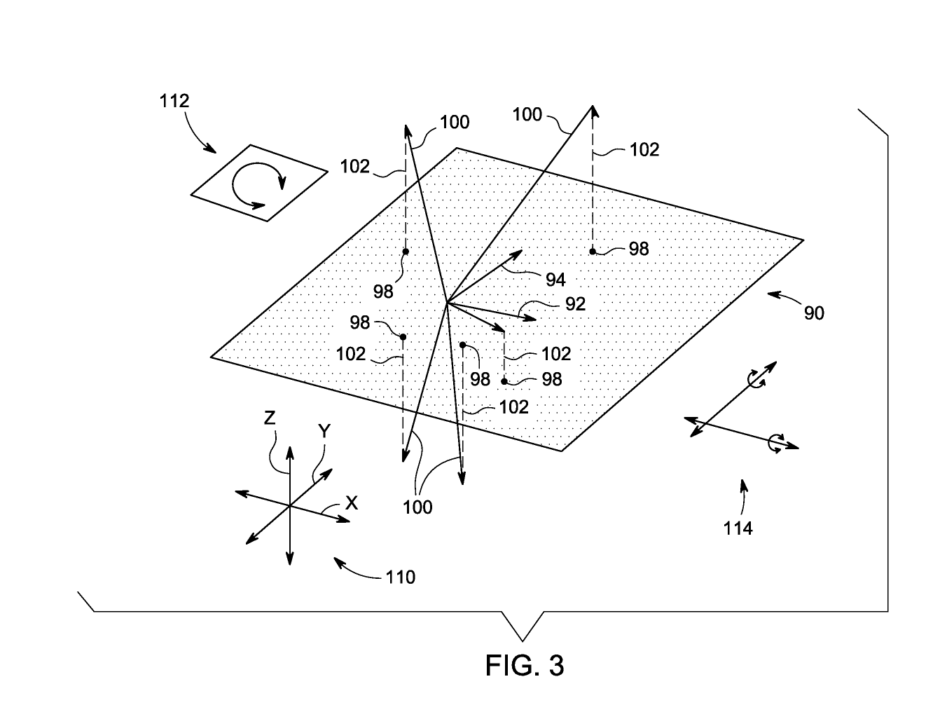

Turning to FIG. 3, a plane 90 is depicted which is defined by a two-axis (depicted as axes 92 and 94) electromagnetic sensor assembly 36 (e.g., an electromagnetic coil assembly or a magnetoresistance sensor assembly). For example, in one embodiment, the sensor assembly 36 may consist of two electromagnetic coils (or other individual sensors) positioned so that each sensor is sensitive along a different axis to provide the two axes of sensitivity. In the depicted example, the two-axes 92, 94 are orthogonal to one another (implying the underlying sensor components are positioned or fixed orthogonal to one another). In other embodiments, however, the axes 92, 94 need not be orthogonal to one another to define the plane 90, though typically the spatial relationship of the axes 92, 94 will be known.

As depicted, the plane 90 (and associated sensor assembly 36) defines a two-dimensional coordinate system (e.g., x and y) with respect to the axes 92, 94, with one axis (e.g., axis 92) defining an x-dimension and the other axis (e.g., axis 94) defining the y-dimension. The signals measured by the sensor are relative to this sensor coordinate system (defined by the x and y axes), and the position/orientation of the sensor assembly is computed from these measurements. Positioning of the sensor assembly 36 in the larger context of the navigation system 10 may be defined or described based on a position of the plane 90 (i.e., centered about the intersection of the axes 92, 94) and by an orientation of the plane 90 (i.e., how the plane is rotated or tilted) with respect to a defined coordinate system that may be defined for the volume in which the sensor assembly 36 is being navigated. For example, the three-dimensional position of the sensor assembly 36 may be defined in terms of x, y, and z coordinates within a volume for which those dimensions have been defined. In one embodiment, the x, y, and z axes may be defined relative to the transmitter assembly. Likewise, the orientation of the sensor assembly 36 may be defined as the roll, pitch, and yaw (such as of plane 90) within the same three-dimensional volume.

When exposed to one or more electromagnetic fields, a measurement 98 may be made for each electromagnetic field. With respect to FIG. 3, a given electromagnetic field line at a given location within the volume may be characterized as a 3D vector 100, where the starting point of the vector is at the origin of the two-dimensional plane 90 (i.e., at the intersection of the axes 92, 94), and in which the length of the vector 100 indicates the local strength of the respective electromagnetic field and the direction of the vector 100 indicates the respective local direction or orientation of the respective electromagnetic field. Different vectors 100 as seen by the sensor assembly 36 may correspond to different transmitter coils of the transmitter assembly 34, where each transmitter coil may transmit at a different frequency so as to be distinguished from the other coils. In the context of the sensor assembly 36, a measurement 98 made by the sensor coils (or other sensing components) that define the plane 90 corresponds to the projection 102 of a respective vector 100 onto the plane 90. That is, the measurements 98 are the two-dimensional coordinates of the projection 102 of a field line vector 100 endpoint onto the two-dimensional plane 90 defined by the sensor axes 92, 94. More specifically, each individual sensor (or coil) provides a measurement that corresponds to the component of the field line in the direction of the sensitivity axis associated with that sensor (or coil). In combination, these measurements define a point in the two-dimensional plane 90. Based on these measurements, the strength and direction of the measured field may be partially determined based upon coordinates of the measured data in the two-dimensional plane 90. In one embodiment, this processing step is based on the distance of the measurement 98 from the intersection of the axes 92, 94 and the direction of the measurement 98 relative to the intersection of the axes 92, 94.

As will be appreciated, in the context of navigation, changing the position of the sensor assembly 36 in one or more of the cardinal directions 110 is effectively a translation of the coordinate system defined by the axes 92, 94 (which may be considered as corresponding to the respective electromagnetic coils or other sensing components) defining the plane 90. Therefore, since in most locations within the volume the orientation of the field lines is relatively constant, translating the sensor assembly 36 may primarily be viewed as changing the length of the field line vectors 100 as the intersection of the axes 92, 94 is repositioned within the volume.

Similarly, changing the orientation of the sensor assembly by rotating 112 the sensor assembly 36 in-plane involves only a change in the coordinate system. For this specific rotation, however, once the change in coordinates is properly translated, the measurements remain unchanged.

Conversely, rotations 114 of the sensor assembly 36 that are not within the original plane 90 may cause changes in the measurement 98 corresponding to a given field line vector 100. Since a rotation alone does not impact the position of the sensor assembly within the field, the vector 100 representing local field strength and orientation remain unchanged. However, since the coordinate system (defined by axes 92, 94) rotates with the associated sensor assembly, the measured coordinates in the sensor plane 90 may change smoothly, as discussed herein, as the plane 90 is rotated.

With the preceding in mind, the present approach considers the local impact of deviations in the measurements on estimates of position and orientation 98 obtained using a sensor assembly 36. In particular, in certain implementations a locally linear model of the relationship between deviations in the measurements/data and the associated changes in estimated position and orientation measurement is employed. Deviations in the measurements may be associated with measurement noise, small displacements of the sensor assembly, mis-calibration, etc. To address changes or effects related to measurement deviations that may be non-linear in nature, the linear modeling approach may be iterated so that the iterated linear model characterizes the relevant non-linear effects. In one implementation, the local linear model employed in such an update process is:

J·p=n (1)

where J is the Jacobian matrix, p is the position and orientation vector (e.g., p may be a 6-dimensional vector, with three components relating to the position of the sensor assembly, and three components relating to the orientation), and n is the corresponding vector representing the deviation in the data/measurements, where the measurements are the coordinates of the points 98 in the plane 90. More accurately, n represents a deviation in the data, and p represents a change in position and orientation, and their relationship at a given location/orientation of the sensor assembly may be modeled/approximated by the linear relationship defined by equation (1). This relationship may be used for iterative improvement of the current estimate of the position/orientation of the sensor within the volume, and it may also be used to model/predict system behavior, sensitivity to noise, etc.

With the preceding discussion in mind, the current approach iteratively estimates position and orientation of a sensor assembly 36 (and thereby estimates position and orientation of the tool tip 52) so that the distance to the measurements 98 is minimized in the L2 sense (i.e., in the sense of the Euclidean distance between coordinates in the sensor plane 90). Typically the process begins by using an estimate of the position and orientation of the sensor assembly 36, i.e., a seed point, that is then iteratively updated until the goodness of fit is maximized. One suitable algorithm that may be employed in conjunction with this approach is the Levenberg-Marquardt algorithm:

[j^{T}·W·j+κ²·l]·p=j^{T}·W·n (2)

Where n is the current difference to the observed/measured data, J is the Jacobian associated with the current position/orientation estimate, W is an optional weight parameter/matrix, and κ² is a regularization parameter that may be adjusted to ensure convergence of the iteration. The vector p is the resulting update to the current position/orientation estimate and is obtained by solving equation (2). The current estimated position/orientation is updated correspondingly and the process is iterated, while κ is selected based on the convergence behavior of the iterative process. For example, if the goodness-of-fit deteriorates as the Levenberg-Marquardt algorithm is iterated, κ, and thereby κ², may be increased. In practice, this means that the parameter κ may be updated based purely on the convergence behavior of the algorithm, without any regard to the local specific characteristics of the Jacobian (which in turn depends on the local magnetic fields and the position/orientation of the sensor assembly). In particular, if the Jacobian is not near-singular, the added regularization factor κ² may drive the iteration toward the wrong solution. However, if the Jacobian is near singular, κ² regularizes the solution, though it may impact even large singular values if not chosen appropriately, as discussed herein.

In an iterative application of the Levenberg-Marquardt algorithm, each iteration of the algorithm may be considered to represent the solution of a Tikhonov-regularized problem. As will be appreciated, Tikhonov regularization is an approach for the regularization of ill-posed problems, such as problems where a solution is not unique or doesn't exist. For example, given a system of linear equations, represented by the matrix A (which may be singular), the problem may be regularized by adding a penalty on the norm of the solution x, and determining x such that:

∥Ax−b∥²+∥Kx∥²→min (3)

[symbol]

x=(A^{T}A+K^{T}K)⁻¹A^{T}b

The solution x may be found (as indicated above) which keeps both the residual error of the system of equations, and the L2 norm (∥Kx∥²) small. Note that this solution is identical to the solution of a single Levenberg-Marquardt iteration, with J=A. With K=κ·l (i.e., κ is a multiple of the identity matrix) and the singular value decomposition A=U·Λ·V^{T}, for singular values λ (which are given by the diagonal values of matrix Λ) the solution x of the Tikhonov regularized problem may be written in the form:

[symbol]

x=V·D·U^{T}·b (4)

where D is a diagonal matrix having elements:

d=λ/(λ²+κ²)

where κ is the regularization factor that may be defined by the user (or that may be automatically updated, e.g., as a function of the convergence of the iteration, in the context of the iterative process). Where the use and the value of the regularization factor κ is appropriate, this term has a minimal impact on large singular values λ but functions to keep small singular values A, from having a disproportionate impact on the solution. As will be appreciated, based on the present discussion, algorithms or system implementations may take into account the position and/or orientation of a sensor assembly 36, and the Jacobian at that position and orientation, in setting the regularization parameter κ in the Levenberg-Marquardt iteration to an appropriate value. That is, based on the known field properties and the known (or currently estimated) position and orientation of the sensor assembly 36, the regularization parameter κ may be set to a suitable value. In one embodiment, the regularization parameter κ is updated for each iteration step, as the estimated position and orientation are updated.

For an indication of how ill-posed a given problem is, and thus how appropriate the use of a regularization factor κ may be and how it may be selected, a condition number may be determined that is the ratio of the largest singular values λ to the smallest singular values λ (i.e., the condition number=λₘₐₓ/λₘᵢₙ). Analysis of the condition number obtained for a representative system at different x, y locations at different heights (i.e., z=15 cm and 30 cm) at different orientations (e.g., 28 different orientations) indicates that the condition number varies strongly depending on location and orientation of the sensor assembly 36. Based on the condition number, an appropriate regularization parameter κ may be selected that is either less than λₘᵢₙ (in case the condition number is small), or that is between the largest and the smallest singular value (if the condition number is large). In one embodiment, the regularization parameter κ in the Levenberg-Marquardt iteration is updated according to these rules, where the maximum and minimum singular values are determined as a function of the currently estimated position and/or orientation of the sensor assembly. In one embodiment the regularization parameter may be updated for each individual iteration step, while in another embodiment the parameter is updated periodically. In yet another embodiment, appropriate regularization parameters may be precomputed for sub-regions of the covered volume, and the regularization parameter is selected according to the subregion the current estimate of the sensor position/orientation is located in. In a further embodiment, other appropriate iterative strategies (different from Levenberg-Marquardt) are used, where e.g., an additional stepsize is selected (in addition to the regularization parameter κ that controls the trade-off between large and small singular values); or, where different update strategies are chosen for subspaces corresponding to the columns of the matrix V (i.e., combinations of positions/orientations that correspond to small singular values may be treated differently than combinations of positions/orientations that correspond to large singular values), etc.

The condition number also provides a measure as to how unbalanced the response or impact to noise is at different positions and orientations within the navigation space, with some noise components having a much stronger impact on the result than others. In particular, the condition number quantifies the relative impact of different noise components, though it does not quantify the base-level noise impact, which must be otherwise determined This observation motivates the use of separate parameters for regularization and stepsize, as discussed above. Further details on strategies for managing noise are provided herein below.

Further, in certain implementations, new seed points for the tracking algorithm may be periodically recalculated or reset based upon predicted values from the current and/or previous estimates of position and orientation. In such an embodiment, the predicted position and orientation may be determined using the most recent position and orientation measurement. In one embodiment, the selection of a new seed point may involve using an estimated speed derived using some temporal subset of recent position and orientation measurements. By way of example, the most recent result of an iteration of the tracking algorithm may serve as the seed for the next iteration of the tracking algorithm.

With the preceding in mind, the characterization of the impact of measurement deviations (noise, and other errors) on the position and orientation estimates, and the associated measurement approaches discussed herein, may be used in a variety of contexts, such as to improve navigation system performance. For example, in one implementation, a suitable position and orientation determination algorithm, such as the Levenberg-Marquardt algorithm, may be employed in an embodiment that enables a mode of operation as a self-calibrating navigation system. In one such embodiment, instead of (or in addition to) employing the conventional L2 error for goodness-of-fit determination, measurement data and/or error may be expressed in a polar coordinate system. That is, instead of decomposing measurements 98 into an x, y coordinate system, measurements 98 may instead be decomposed into polar coordinates (i.e., angle and amplitude).

In such an approach, error can be weighted differently than in conventional approaches. For example, in such a system, error can be weighted in terms of angle versus distance (i.e., radius). As will be appreciated, in such an implementation, if the weight associated with the distance is zero (i.e., zero amplitude), the tracking system is completely decoupled from field strengths and relies solely on local field orientations. Such an approach may be particularly useful in circumstances where the localized field lines for a given field are sufficiently separated in orientation and where the field strength is known to be (or expected to be) good. In such an approach, the measured orientation (direction in the context of a polar coordinate system) alone may be used to derive the desired tracking information for the sensor assembly 36 with respect to that field. That is, in such an implementation, both position and orientation for the sensor assembly 36 (and associated tool tip) may be determined for a field using only the measured direction data (i.e., direction of the measured coordinates within the coordinate system associated with the sensor plane) for that field.

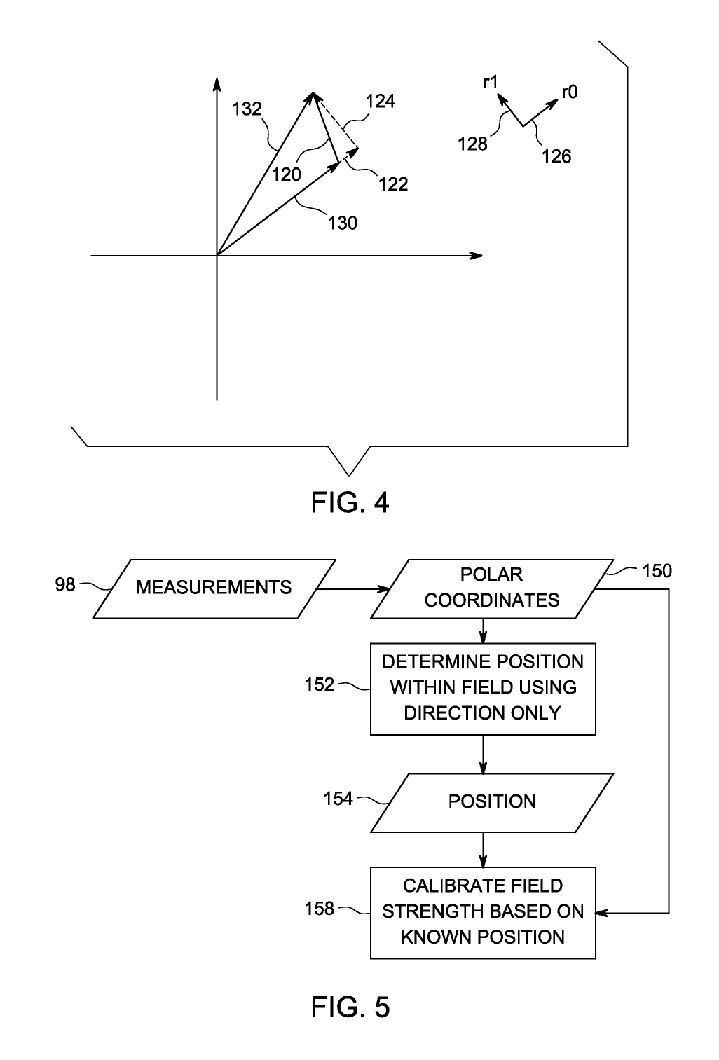

Turning to FIG. 4, in one such embodiment, the vector n 120 of data deviations (determined using the expected measurement 130 (based on the current estimate of position and orientation) and the true measurement 132) is pre-multiplied with a matrix representing a decomposition of each pair of measurements into a component 122 along the direction of the measured data, and a component orthogonal 124 to it. The component 122 along the (projected) field direction may be completely discarded, or weighted lower than the component 124 orthogonal to it, thereby enhancing the relative importance of the data corresponding to the direction of the field lines over the data corresponding to the field strength. For example, the modified equation Γ·R·j_19 p=Γ·R·n may be used as the basis for building an iterative strategy (similar to how, in the discussion above, the equation j·p=n was used to derive the iterative update (2)) for determining the position and orientation of the sensor assembly. Here, the matrix R denotes a change of coordinate systems for each pair of measurement values corresponding to a single field, such that one component in this new coordinate system corresponds to the direction of the measurement data, and the other component corresponds to the direction orthogonal to it. The matrix Gamma (Γ) is a weighting matrix, which gives relative weights to the different components of the data. For example, the matrix R may be block-diagonal, where each block consists of a 2×2 matrix, and the first row of each block consists of a unit-vector r₀ 126 in the direction of the measurement, and the second row corresponds to a unit vector r₁ 128 orthogonal to it (as illustrated in FIG. 4). In this case the matrix Gamma may be diagonal, with all even-indexed entries being set to 1, and all odd-indexed entries set to 0, thereby weighting the error in the direction of the measurements with a weight of zero, thereby relying only on the orientation of the field lines. By adjusting the weights in the matrix Gamma, one may, for example, balance between this “orientation-only” strategy as outlined here, and a more traditional strategy (all diagonal elements of Gamma being set to 1). Note that these different strategies may also be balanced differently for different fields, or as a function of the position within the volume. In one embodiment, the weight for any measurement that is close to the origin is weighted with 0 (for both components), since in the presence of noise the direction of the measurement may not be reliably determined

Such an approach may allow for a variety of benefits, including allowing auto-calibration of the system, allowing detection of mis-calibration of the system, or more generally, allowing for consistency checks (e.g., field distortion and so forth) based on detection of inconsistencies between the angular and distance error metrics. For example, with respect to auto-calibration, positioning the sensor assembly 36 at a location for a given field and computing position and orientation information based solely on direction (or angle) information would allow for a determination of the field strengths from the measurements, i.e., a calibration of the field strength measurement using the sensor assembly 36. In this manner, the field strength may be calibrated by guiding the sensor assembly to a known location (or within a known subregion of the volume), where the position/orientation of the sensor are derived from the orientation data alone, and the field strength for each field may then be derived from the measurements, thereby calibrating the system field strengths.

For example, as depicted in FIG. 5, a measurement 98 may be obtained using the sensor assembly 36. In one embodiment, this measurement may be expressed in polar coordinates 150 (i.e., a direction or angle and an amplitude). Based on the direction component and on the known or expected field characteristics, the position 154 of the sensor assembly 36 may be determined (block 152). Based on this position 154, the field strength may be determined thereby providing a field strength parameter in the automatic or self-calibration (block 158) of the tracking system.

Similarly, such an approach may be used to detect mis-calibrations and/or drifts in field strength. For example, a position and orientation of the sensor assembly 36 may be determined based on orientation alone and the measured field strength at that location may then be compared to the expected field strength for that location within the field. A difference between the expected and observed field strength may be indicative that the tracking system (and in particular the field strengths) is mis-calibrated and a notification may be provided to the user. Generally, in a well-calibrated system, the position/orientation estimation performance will be superior if both orientation and field strength are used in the processing. Therefore, in another embodiment, the position and orientation are derived using both orientation and field strength, and the residual error is analyzed. If there is a systematic component in the residual error, this may indicate a mis-calibration of the system. In yet another embodiment, the directions of the measurement data is evaluated and compared against the expected directions based on the orientations of the field lines at this position/orientation. If there is a systematic error in this direction (or angular component of the measurements), this may indicate a field distortion and a notification may be provided to the user.

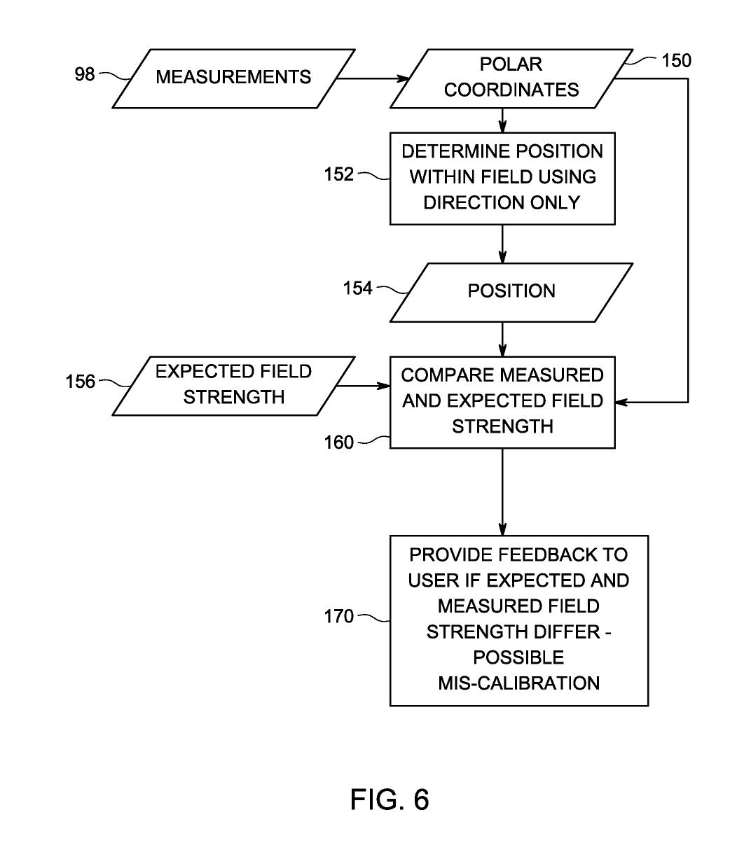

For example, as depicted in FIG. 6, a measurement 98 may be obtained using the sensor assembly 36. In one embodiment, this measurement may be expressed in polar coordinates 150 (i.e., a direction or angle and an amplitude). Based on the direction component and on the known or expected field characteristics, the position 154 of the sensor assembly 36 may be determined (block 152). Based on this position 154 an expected field strength 156 may be determined and compared (block 160) to the measured field strength determined from the measurement 98. Difference between the measured and expected field strength beyond some defined tolerance or threshold may result in a determination that the sensor assembly 36 is mis-calibrated. In such a circumstance, feedback (such as a visual and/or audible indication) may be provided (block 170) to the user. In one embodiment, if for example the field strength corresponding to the measurements 98 is systematically lower than the expected field strength 156 (for this field), then a corresponding message is displayed for the user. In another embodiment, both measured direction and strength are used for the estimation of position and orientation, while in parallel a decomposition of the measurement data (or the residual error) in direction and angle is performed. This decomposition is used to detect mis-calibration and similar inconsistencies, while the main position/orientation computation benefits from the better accuracy of using both components.

It should also be appreciated that, in view of the position and orientation determination approaches discussed herein, a navigation system 10 may be provided that provides feedback to a user (such as via display 42) related to a self-calibration procedure (as well as guiding the calibration procedure itself), which may involve moving the sensor assembly 36 within or to a defined region or band until self-calibration is completed. Such a notification and self-calibration may be based on internal consistency checks and error estimates performed by the system 10 and may involve instructions to move the sensor assembly 36 to a given location, such as a location of known field orientation, to facilitate the calibration process. The process may also involve feedback about remaining calibration time (until the desired calibration accuracy is achieved), additional calibration locations/regions, and so forth. In one embodiment, an auto-calibration process may be performed concurrently while a procedure is being performed, and position and orientation data are computed and provided to the user. The determination of the position and orientation may be, for example, performed in a robust mode which relies only on the direction of the measurement data. A notification about the currently used robust mode may be provided to the user.

In other implementations, the system may leverage the knowledge of the characteristics of the noise contributions to estimated position and orientation (as discussed in more detail herein below) at different sensor assembly positions and orientations. For example, in one embodiment, the navigation system, based on the position and orientation of the sensor assembly 36 and the associated noise characteristics, may multiplex and/or combine frequencies between transmission coils so as to generate more data points (i.e., measurements 98) for the more useful coils. For instance, in circumstances where the sensor assembly 36 is high or otherwise distant relative to the transmitter assembly 34 (e.g., where z is large), a limited number of transmitter coils within the transmitter assembly may drive overall performance. Similarly, at other locations along, e.g., the periphery of the navigation space, overall performance may be driven largely by a limited number of transmission coils of the transmitter assembly. In such circumstances, it may be useful to switch off those transmission coils that are not contributing useful position and orientation measurements and to operate the remaining coils in a multiplexed manner so that they alternately or simultaneously transmit at their original frequency as well as at the frequency of a coil that is switched off

By way of example, assuming two coils are being initially operated, one at a first frequency and the other at a second frequency. For those locations in the navigation space where the first coil provides useful position and orientation measurements but the second coil does not, the second coil may be switched off and the first coil may be operated alternately or simultaneously at both the first and second frequency so as to produce measurements 98 at both frequencies. In this manner, a transmission coil that is providing useful signal may generate multiple measurement points, one for each frequency at which it is being operated. Similarly, in other implementations field strengths for one or more of the transmission coils may be adjusted (e.g., increased) when the sensor assembly 36 is determined to be in an ill-conditioned location and/or orientation (e.g., where signal is poor or noise is high). The decision for switching coils on or off may be based on distance to the coils, local field strengths associated with those coils, as well as local orientation of the field lines from those coils, or suitable combinations of these criteria.

With the preceding discussion relative to the derivation of the algorithm for computing the position/orientation in mind, the singular value decomposition (SVD) of the Jacobian may also be used to yield the noise transfer function where:

j=U·Λ·V^{T} (6)

The deviation p in the position/orientation due to a noise term n (in the measurements) may be determined (from equation (1)) as

p=V·Λ⁻¹·U^{T}·n=V·Λ⁻¹·n′ (7)

where U and V are orthogonal matrices, Λ is a diagonal matrix containing the singular values, and Λ⁻¹ is the inverse diagonal matrix (which provides weighting and scaling factors), T indicates the transposition of a given matrix, and where U^{T}·n gives a modified noise term n′. The vector n is a noise vector (due to noisy measurements) which is assumed to be white Gaussian noise with independent components and uniform standard deviation. Therefore n′ represents also independent white Gaussian noise and has the same standard deviation as n. Thus, for a known field and sensor position/orientation, if the SVD of the Jacobian is known or determined, it is possible to determine what positions and orientations of the sensor assembly 36 will be more sensitive to noise. It may be assumed, in certain implementations, that the measurement noise, n, is independent of other considerations and may be characterized using a Gaussian distribution. In such circumstances, the SVD of the Jacobian matrix yields a noise transfer function that can account for changes in translation and rotation of the sensor assembly 36 that are observed as a function of the measurement noise. Such information can be leveraged to determine what happens to a measurement 98 in the presence of noise at particular positions and orientations of the sensor assembly 36. For example, it may be determined what combinations of positions and orientations of the sensor assembly 36 are more sensitive to noise and this information may be used to improve system or procedure performance.

With the preceding in mind, a position error entitlement analysis was performed using noise/error propagation analysis. With respect to the noise model, independent Gaussian noise was assumed to be present and to act on each measurement component and to have the same standard deviation. Based on this, relative standard deviation was modeled or predicted for each x, y, z-position. The analysis was presumed to be valid for low noise levels, where the locally linear model was assumed to be sufficiently accurate. In a first study, for z-values of 15 cm and 30 cm in height (i.e., the plane 90 in which measurements were generated was 15 cm or 30 cm above the transmitter coil surface) sample measurements were generated for ±20 cm in x, y with 5 mm sampling. In this sampling protocol, 28 different orientations of the sensor plane 90 were considered at each of the two z-values, with standard deviation (i.e., error) measurements derived for each sample location. In one instance, the average position error (i.e., standard deviation) was determined at each x, y, z position by averaging across all orientations at each sample point. In the other study, instead of averaging the error at each x, y, z position for the range of orientations, only the worst error value (i.e., the error value at the worst orientation) was kept, thereby providing a “worst case” position error study.

Based on these studies, it was observed that error or noise propagation could be modeled as a locally linear phenomena. Further, it was observed that position error at 30 cm height relative to the transmitter coil was more significant than the position error observed at 15 cm height, assuming a constant noise model. That is, height above the transmitter coils was a significant contributor to measurement error. In addition, the worst-case error (i.e., the observed error for the worst-case sensor assembly orientation at a given x, y, z location) was observed to be twice as large relative to the corresponding average error near the edges of the measurement region of interest. That is, orientation could be a large contributor to measurement error with unfavorable orientations of the sensor assembly having an increased sensitivity to measurement noise.

In addition to the system improvements noted above, the present approaches may also be employed to improve signal processing algorithms associated with position and orientation determination. For example, to improve accuracy, a smoothing or update type process may be employed on one or both of the raw measurement data or on the position and orientation data generated as discussed herein. By way of example, a Kalman-filter (or similar filter type) which essentially processes a moving window of time series data points or estimates of the current position/orientation based on previous estimates combined with the current measurements, may be employed to provide suitable temporal smoothing of the raw measurement data and/or of the generated position and orientation data points. Such an approach may improve the accuracy of the results and provide a better estimate of the underlying system state, here the true location and orientation of the sensor assembly 36 at a given time.

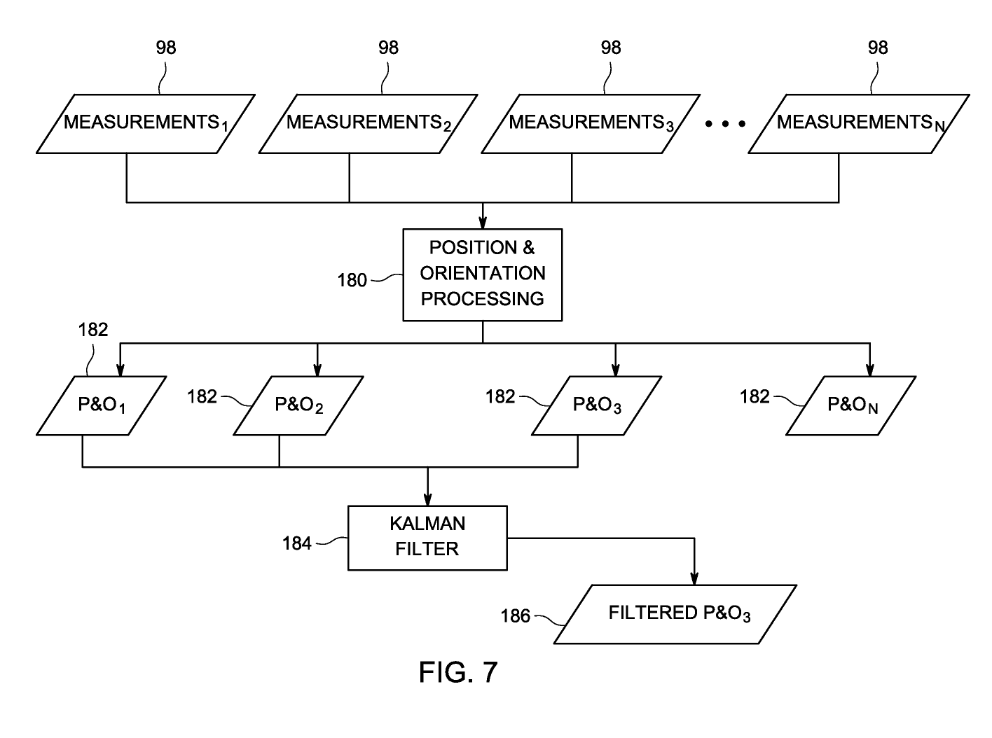

By way of example, turning to FIG. 7, a series of measurements 98 acquired over time may be processed (block 180) in accordance with the position and orientation algorithms discussed herein to derive a corresponding time series of positions and orientation values 182 for a given sensor assembly 36. In one implementation, a moving window of the time series of position and orientation values 182 may be provided as inputs to a Kalman-filter 184 or other suitable temporal smoothing filter. In such an implementation, based upon each provided window or series of data, a corresponding temporally-smoothed data value 186 is generated. In this example, position and orientation values P&O₁, P&O₂, and P&O₃ are provided as inputs to the Kalman Filter 184, which generates a temporally-smoothed value 186, filtered P&O₃, using the provided time-series of data points. In such a process, the next window of initial position and orientation values 182 (i.e., P&O₂, P&O₃, and P&O₄) would then be provided to the Kalman filter to generate the next temporally smoothed value 186 (i.e., filtered P&O₄), and so forth. As noted above, such an approach may be applied at multiple points within the overall algorithm. For example, temporal smoothing may be applied to the raw measurement data 98 prior to calculation of the position and orientation values 182 and may again be applied to the generated position and orientation values 182.

In other embodiments, adaptive smoothing may be employed, such as to smooth the displayed or apparent motion of the tool 12 incorporating the sensor assembly 36. Such adaptive smoothing may employ one or more smoothing parameters that may be modified or set based on the speed of the sensor assembly 36 within the observed navigation space. That is, successive position and orientation values, determined at known times and in accordance with the approaches discussed herein, may be used to determine the speed of the sensor assembly 36. This calculated speed may in turn be used to set the smoothing parameters to be applied. For example, the slower the sensor assembly 36 moves, the greater the degree of smoothing that may be applied.

While sensor speed is one factor that may be used to determine the degree of smoothing to be applied at a given time, other factors may also contribute to the degree of smoothing applied. For example, in one embodiment that takes into account noise considerations as discussed herein, stronger smoothing parameters (e.g., greater smoothing) may be applied for combinations of positions and orientations where the data is expected to be noisier (i.e., for regions associated with smaller singular values). Similarly, components of the position/orientation estimate that are more sensitive to noise (i.e., the components that are associated with small singular values of the Jacobian) may be smoothed stronger than other components. Note that equation (7) allows components to be identified in the measurement noise that contribute disproportionally to the position/orientation error (i.e., the components associated with a small singular value of the Jacobian), and conversely, the combinations of positions and orientations that are most susceptible to noise. All this information, as well as known noise characteristics, etc., may be used appropriately in the filtering/smoothing of the measurement data and/or the position/orientation estimates. In one embodiment, one parameter that is associated with the smallest singular value of J is smoothed more strongly than other parameters. This parameter may be given by the combination of variations/deviations in position/orientation corresponding to the column vector of V that is associated with the smallest singular value of J.

In another example, jump or discontinuity detection processing may be employed to provide an indication as to discontinuities in the motion data, which may result in greater smoothing being applied before and after such events, but not during those events. Likewise, secondary motion data from other physiological monitoring systems, such as electrocardiograms or measured respiratory data, may provide information regarding cardiac or respiratory phase that may be accounted for in a smoothing process. For example, increased or enhanced smoothing may be applied during cardiac or respiratory phases associated with smaller physiological movement. Note that smoothing may be applied to the orientation/position directly, as well as to derived parameters, such as speed, and so forth. The characteristics of the smoothing operation may be adapted, e.g., to the current physiological state (e.g., heart in diastole vs. systole, different smoothing during a breath-hold, etc.). Similarly, other adaptations or constraints, such as based on the vascular pathway derived from imaging data or known limitations on the flexibility or bending of the tool, may be incorporated in such adaptive processing so as to rule out unlikely or physically impossible scenarios. It should be appreciated that for purposes of auto-calibration, detection of mis-calibration and similar consistency checks, the smoothing/integration may be performed over longer time intervals than for normal operation (estimation of position/orientation). As discussed previously, both processes (auto-calibration/consistency checks, and position/orientation estimation) may be performed in parallel, each having their own separate and distinct smoothing parameters.

Further, aspects of noise processing determination and processing, as discussed herein, may be used in either the temporal smoothing (e.g., Kalman filter operations) or adaptive smoothing. For example, for positions and/or orientations within a given field where increased noise is expected to be present (such as at greater distance or height from the transmit coil), a longer integration interval may be employed. For instance, in the case of temporal smoothing employing a Kalman filter, a wider temporal window may be employed to provide a longer data integration interval. In addition, when operating within a portion of the navigation space known or expected to have noisier conditions (such as at greater distances from a transmit coil) an indication (e.g., feedback) may be provided to the operator (such as via display 42) to slow down, allowing more measurements 98 to be acquired in the noisy region and thereby improving useful signal. Similarly, in implementations where adaptive smoothing is employed, one factor that may determine the degree or extent of smoothing employed at a given time or location is the observed or expected noise characteristics for the sensor assembly 36 at a given position and orientation with respect to a given field. For example, stronger smoothing may be applied when data is observed to be or expected to be noisy. These parameters may also be adapted to a specific accuracy target (e.g., when navigation accuracy is required to be within 1 mm), etc. As discussed earlier, smoothing parameters and so forth may be determined as a function of the characteristics of the Jacobian at the current estimated position/orientation (in combination with other prior knowledge), or some or all of these parameters may be pre-computed, e.g., for subregions within the volume, in which case they may be selected, e.g., using a look-up table based on the current position/orientation estimate.

It should also be appreciated that, in view of the position and orientation determination approaches discussed herein, a navigation system 10 may be provided that provides feedback to a user (such as via display 42) related to the positional accuracy and/or error amplitudes for the tool 12. For example, in view of the preceding discussion, based on a given position and orientation of a sensor assembly 36 within the navigable volume, a color coded or quantified metric may be displayed for a user to convey a degree of confidence in the positional accuracy of the tool 12 and/or to convey the expected error amplitudes (e.g., margin of error) associated with the displayed position and orientation information. In one embodiment, feedback is provided to the user if the noise and/or expected accuracy exceeds predefined thresholds. Similarly, as discussed below, feedback may be displayed to instruct the operator to proceed more slowly, such as to improve the position and orientation determination within noisy regions and/or to maintain operation within defined error bounds. In another embodiment, the user may be notified that rotating the tool/sensor may result in better performance. This feature may also be used in surgical planning, i.e., the trajectory of a device may be selected/optimized beforehand such that the expected navigational accuracy is maximized.

Technical effects of the invention include improvement of position and orientation tool tracking in a medical navigational system. In one embodiment, a position of a surgical or interventional tool may be determined using the orientation or field direction data, that is, position may be determined independent of field strength or magnitude. Feedback or indications of a mis-calibration may also be provided to a user based on position information determined independent of field strength or magnitude. Likewise, in certain embodiments, the navigational system may be auto-calibrated using position information determined independent of field strength or magnitude.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A surgical or interventional navigation system, comprising:
a transmitter assembly comprising at least a first transmitter coil and a second transmitter coil;
a sensor assembly comprising one or more sensor components defining a plane; and
an electromagnetic tracking system in communication with both the transmitter assembly and the sensor assembly, wherein the electromagnetic tracking system is configured to:
acquire a plurality of measurements using the sensor assembly, wherein each measurement corresponds to a projection of a three-dimensional vector onto the plane;
determine one or both of an orientation and a position of the sensor assembly based on the polar coordinates of the measurements.

2. The surgical or interventional navigation system of claim 1, wherein the electromagnetic tracking system is further configured to auto-calibrate the electromagnetic tracking system based on deviations between expected measurements and observed measurements.

3. The surgical or interventional navigation system of claim 1, wherein the electromagnetic tracking system is further configured to detect mis-calibration of the field strengths or electromagnetic field distortions based on deviations between expected measurements and observed measurements.

4. A surgical or interventional navigation system, comprising:
a transmitter assembly comprising at least a first transmitter coil and a second transmitter coil;
a sensor assembly comprising one or more sensor components defining a plane, wherein the sensor assembly is configured to generate measurements corresponding to position and orientation of the sensor assembly within electromagnetic fields generated by the transmitter assembly; and
an electromagnetic tracking system in communication with both the transmitter assembly and the sensor assembly, wherein the electromagnetic tracking system is configured to:
drive the first transmitter coil at a first frequency and the second transmitter coil at a second frequency when the sensor assembly is within a one or more of a threshold distance, field strength, or field orientation relative to both the first transmitter coil and the second transmitter coil; and
drive the first transmitter coil in a multiplexed manner at the first frequency and at the second frequency and not drive the second transmitter coil when the sensor assembly is within the threshold distance, field strength, or field orientation relative to the first transmitter coil and outside the threshold distance, field strength, or field orientation relative to the second transmitter coil.

5. The surgical or interventional navigation system of claim 4, wherein the electromagnetic tracking system drives the first transmitter coil in a multiplexed manner at the first frequency and at the second frequency by alternately driving the first transmitter coil at the first frequency and the second frequency.

6. The surgical or interventional navigation system of claim 4, wherein the electromagnetic tracking system drives the first transmitter coil in a multiplexed manner at the first frequency and at the second frequency by simultaneously driving the first transmitter coil at the first frequency and the second frequency.

7. A surgical or interventional navigation system, comprising:
a display;
a transmitter assembly comprising at least a first transmitter coil and a second transmitter coil;
a sensor assembly comprising one or more sensor components defining a plane, wherein the sensor assembly is configured to generate measurements corresponding to position and orientation of the sensor assembly within electromagnetic fields generated by the transmitter assembly; and
an electromagnetic tracking system in communication with the transmitter assembly, the sensor assembly, and the display, wherein the electromagnetic tracking system is configured to provide feedback comprising user instructions via the display, wherein the feedback is based on the position and orientation of the sensor assembly within a navigable volume defined by the transmitter assembly and on the expected noise characteristics at the position and orientation.

8. The surgical or interventional navigation system of claim 7, wherein the feedback comprises instructions to adjust one or both of the speed at which the sensor assembly is being moved or the rotation of the sensor assembly based on the current position and orientation of the sensor assembly.

9. The surgical or interventional navigation system of claim 7, wherein the feedback comprises an indication of the current calibration state of the sensor assembly.

10. The surgical or interventional navigation system of claim 9, wherein the indication includes one or more instructions for enabling an auto-calibration during a navigational procedure.

11. The surgical or interventional navigation system of claim 7, wherein the feedback comprises a metric that conveys one or both of a degree of confidence or an expected error amplitude in the positional accuracy of the sensor assembly, wherein the metric is based at least in part on the position and orientation of the sensor assembly within a navigable volume defined by the transmitter assembly.

12. The surgical or interventional navigation system of claim 7, wherein the feedback comprises an indication that the region currently being navigated is subject to measurement noise beyond the threshold level.

13. A method for operating a medical navigation system, comprising:
acquiring a measurement using a sensor assembly, wherein sensing elements of the sensor assembly define a planar measurement space and wherein the measurement corresponds to a projection of a three-dimensional vector onto the planar measurement space;
expressing the measurement in polar coordinates;
determining one or both of an orientation and a position of the sensor assembly based on the polar coordinates of the measurement; and
adapting operation of the medical navigation system or a procedure being implemented using the medical navigation system based on the position and orientation of the sensor assembly.

14. The method of claim 13, wherein adapting operation of the medical navigation system or the procedure comprises adapting the temporal integration or smoothing of at least one parameter based on the position and orientation of the sensor assembly.

15. The method of claim 13, wherein adapting operation of the medical navigation system or the procedure comprises adapting properties of the processing to the speed at which the sensor assembly is being moved.

16. The method of claim 13, wherein adapting operation of the medical navigation system or the procedure comprises determining a degree of mis-calibration based on the position and orientation of the sensor assembly and adapting operation to the degree of mis-calibration.

17. The method of claim 13, wherein adapting operation of the medical navigation system or the procedure to mis-calibration comprises at least one of adaptively calibrating the field strengths and adaptively switching to a computation based predominantly on measurement directions.

18. The method of claim 13, wherein adapting operation of the medical navigation system or the procedure is further based on motion data derived from a physiological monitoring system.

19. The method of claim 13, wherein adapting operation of the medical navigation system or the procedure comprises calibrating the sensor assembly based on an expected electromagnetic field strength at the position

20. The method of claim 13, wherein adapting operation of the medical navigation system or the procedure comprises:
comparing a measured field strength with an expected field strength for the position;
if the measured field strength differs from the expected field strength by more than a specified tolerance, providing an indication of mis-calibration to a user.
